(19) United States
(12) Patent Application Publication
(10) Pub. No.: US 2025/0366729 A1
(43) Pub. Date: Dec. 4, 2025

(54) CORRELATING PULSE-OXIMETRY WAVEFORM SIGNALS WITH BLOOD PRESSURE

(71) Applicant: Guy P. Curtis, San Diego, CA (US)

(72) Inventor: Guy P. Curtis, San Diego, CA (US)

(21) Appl. No.: 19/296,803

(22) Filed: Aug. 11, 2025

Related U.S. Application Data
(63) Continuation-in-part of application No. 18/336,987, filed on Jun. 17, 2023.
Application No. 18/336,987 is a continuation-in-part of application No. 17/496,052, filed on Oct. 7, 2021.
(60) Provisional application No. 63/172,270, filed on Apr. 8, 2021.

(51) Int. Cl. A61B 5/0295; A61B 5/00; A61B 5/024; G16H 40/63; G16H 40/67

(52) U.S. Cl. CPC A61B 5/0295; A61B 5/02416; A61B 5/02438; A61B 5/6824; A61B 5/7235; A61B 5/742; G16H 40/63; G16H 40/67

(57) ABSTRACT

An electronic device that monitors blood pressure in vasculature of an individual is described. This electronic device may perform the operations of: monitoring blood flow of the individual (e.g., using a pulse oximeter); calibrating the blood flow based at least in part on a predefined or predetermined comparison of the blood flow and blood pressure, e.g., in the individual's vasculature; and providing the calibrated blood flow of the individual as a function of time, where the calibrated blood flow indicates at least the blood pressure or a blood-pressure reading for the individual.

### CROSS REFERENCE TO RELATED APPLICATIONS

This application is a Continuation-in-Part of U.S. Non-Provisional application Ser. No. 18/336,987, “System and Method for Correlating Pulse Oximetry Waveform Signals with Blood Pressure,” filed on Jun. 17, 2023, by Guy P. Curtis, which is a Continuation-in-Part of U.S. Non-Provisional application Ser. No. 17/496,052, “System and Method for Correlating Pulse Oximetry Waveform Signals with Blood Pressure,” filed on Oct. 7, 2021, by Guy P. Curtis, which claims benefit to U.S. Provisional Application Ser. No. 63/172,270, “System and Method for Correlating Pulse Oximetry Waveform Signals with Blood Pressure,” filed on Apr. 8, 2021, by Guy P. Curtis, the contents of each of which are herein incorporated by reference.

### FIELD

The described embodiments relate to systems and methods for monitoring blood pressure and blood flow. More particularly, the described embodiments relate to systems and methods that provide comprehensive information regarding the efficacy of an individual's heart muscle function. The disclosed embodiments particularly, but not exclusively, useful for periodically calibrating an oximeter with contemporaneous blood pressure measurements (which may be acquired by a sphygmomanometer) to display information (e.g., periodically, as-needed or continuously) from the pulse oximeter regarding an individual's heart rate, blood flow and/or blood pressure.

### BACKGROUND

A pulse oximeter is a medical device that is used to accurately indicate an individual's local blood-flow characteristics. Notably, a pulse oximeter can record the sinusoidal characteristics of a blood-flow waveform that provide both temporal and amplitude values. For example, the characteristics of blood flow may include the magnitudes of sequential peak amplitudes and the time interval between these peak amplitudes in the blood-flow waveform. With this information, an individual's heart rate and blood-flow volume may be determined. However, these characteristics alone do not indicate another important physical measurement: blood pressure.

In a clinical environment it is often useful to have as much timely information as possible, for both an individual's blood flow and for his/her blood pressure. Collectively, this information is both interdependent and interrelated. However, unlike a pulse oximeter, which can be automatically operated continuously to record a blood-flow waveform, the operation of a sphygmomanometer to measure blood pressure is labor intensive and, realistically, may usually only be employed intermittently. Heretofore, this operational disconnect has, for the most part, been tolerated.

### SUMMARY

An electronic device that monitors blood pressure in vasculature of an individual is described. This electronic device performs the operations of: monitoring blood flow of the individual; calibrating the blood flow based at least in part on a predefined or predetermined comparison of the blood flow and blood pressure, e.g., in the individual's vasculature; and providing the calibrated blood flow of the individual as a function of time, where the calibrated blood flow indicates at least the blood pressure or a blood-pressure reading for the individual.

Note that, during the monitoring, the electronic device may be coupled or attached to the individual, such as a patient.

Moreover, the monitoring of the blood flow may be performed continuously, periodically or as needed.

Furthermore, the monitoring of the blood flow may be performed using a pulse oximeter. For example, during the monitoring, the electronic device may acquire an instance of a pulse oximeter waveform of the individual.

Additionally, the providing may include displaying the calibrated blood flow of the individual.

In some embodiments, the calibrated blood flow may be sinusoidal, with each pulse in the calibrated blood flow having a peak amplitude A. The calibrated blood flow may also have a time interval Δt between the peak amplitude of a given pulse and the peak amplitude of the immediately preceding pulse. Thus, the calibrated blood flow may include a heart rate corresponding Δt and/or a blood-flow volume corresponding to Δt and A. Consequently, in some embodiments, the electronic device may: compute the heart rate and/or the blood-flow volume based at least in part on the calibrated blood flow; and/or may provide the computed heart rate and/or blood-flow volume.

Moreover, the operations may include performing a calibration, where the calibration includes measuring an instance of the comparison of the blood flow and the blood pressure using, at least in part, a sphygmomanometer. For example, the instance of the comparison may be measured at a steady-state condition for the relationship between the individual's blood-flow measurement A and their blood-pressure reading Pmeasured. Note that Pmeasured may be a difference between a systolic pressure and a diastolic pressure. This steady-state condition may then be used by the electronic device to perform the calibration (or to compute the calibrated blood flow, Acalibrated). During the comparison, the electronic device may concurrently obtain a measurement of the blood flow and the blood pressure, e.g., in the steady-state condition. In some embodiments, the predefined or determined comparison of the blood flow and the blood pressure may correlate or include changes in the blood flow ±ΔA with changes in the blood pressure ±ΔP.

Note that, for a constant blood-flow condition, the predefined or predetermined comparison may be expressed as A=P/R, where R is a factor representing the individual's vascular resistance to blood flow. In this ratio relationship, changes in blood flow ±ΔA and changes in blood pressure ±ΔP may be equated to each other for correlation purposes as ±ΔA/Acalibrated ≈±ΔP/Pmeasured. The correlation of blood flow with blood pressure may be made relative to a previously established steady-state condition. For this purpose, the steady-state condition may be expressed as (ΔP)base=Psystolic−Pdiastolic.

Consequently, when providing the calibrated blood flow, the electronic device may display blood-pressure variations ±ΔP corresponding to variations in the blood flow ±ΔA. As noted previously, this correspondence may be made based at least in part on the predefined or predetermined comparison of the blood flow and the blood pressure having a constant (ΔP)base. For the purpose of assessing blood flow in the individual's vasculature, the electronic device may indicate whether there are any consequent changes in heart rate ±Δt that are associated with concurrently measured ±ΔA. Notably, the electronic device may selectively present ±ΔP in the context of either a first operational state wherein Δt is constant, or a second operational state in which Δt is variable.

In the first operational state, Δt may be constant and, to maintain a proper constant blood-flow relationship, R may be varied whenever A is varied in the predefined or predetermined comparison A=P/R. Notably, with a +ΔA there may be a comparable change in R >1, and with a −ΔA there may be a comparable change in R <1. In the short term (such as a few seconds or a few minutes), this relationship may be considered valid because R is anatomically slow to vary. On the other hand, R may become a factor over a relatively longer term.

In the second operational state, Δt may be variable, and R may remain constant to maintain the operational relationship between ±ΔA and ±ΔP. Nonetheless, ±ΔA and ±ΔP may vary somewhat. In this latter case, recalibration may be needed. Thus, in each operational state, the predefined or predetermined comparison may determine a blood pressure P (e.g., for display) that may include clinical information pertinent to the individual's condition.

In some embodiments, the electronic device (or an associated monitor or display) may record variations of ±ΔA and ±Δt in the blood flow during a predefined or predetermined time duration. These measurements may be compared with previous measurements to determine whether ±ΔA and ±Δt have sufficiently stabilized during the predefined or predetermined time duration to identify a new value for the blood flow (e.g., A′). If so, the steady-state condition may be recalibrated. As noted previously, this calibration may be performed (e.g., periodically) using a sphygmomanometer to obtain new blood-pressure readings P′measured to recalibrate a new value for the blood flow A′ as A′calibrated for use with P′measured to identify a new steady-state condition for the individual.

Moreover, in some embodiments, at least some of the aforementioned operations may be performed by a computer system (which may include at least a computer), which may be coupled to the electronic device. For example, the electronic device may provide, to the computer system, the monitored blood flow. In response, the computer system may compute the calibrated blood flow, which is then provided to the electronic device. Thus, the computer system may perform at least sone of the aforementioned operations.

Another embodiment provides an integrated circuit that performs at least some of the aforementioned operations.

Another embodiment provides a computer-readable storage medium for use with the electronic device or the computer system. When executed by the electronic device or the computer system, this computer-readable storage medium causes the electronic device or the computer system to perform at least some of the aforementioned operations.

Another embodiment provides a method, which may be performed by the electronic device or the computer system. This method includes at least some of the aforementioned operations.

This Summary is provided for purposes of illustrating some exemplary embodiments, so as to provide a basic understanding of some aspects of the subject matter described herein. Accordingly, it will be appreciated that the above-described features are examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a block diagram illustrating an example of communication between a monitoring device and an electronic device in accordance with an embodiment of the present disclosure.

FIG. 2 is a flow diagram illustrating an example of a method for providing calibrated blood flow of an individual in accordance with an embodiment of the present disclosure.

FIG. 3 is a drawing illustrating an example of components of a system connected to an individual (such as a patient) in a clinical environment in accordance with the present disclosure.

FIG. 4 is a drawing illustrating an example of a blood-flow waveform of a patient in accordance with the present disclosure.

FIG. 5 is a drawing illustrating an example of a graphical presentation of a tracing profile for the patient's blood flow in accordance with the present disclosure.

FIG. 6 is a drawing illustrating an example of the interactive operation of components in the system in accordance with the present disclosure.

FIG. 7 is a drawing illustrating an example of calibration of a pulse-oximetry waveform in accordance with the present disclosure.

FIG. 18 is a drawing illustrating an example of maximum pressure slope in the aorta as a function of maximum pressure slope in the left ventricle in in a first run in accordance with the present disclosure.

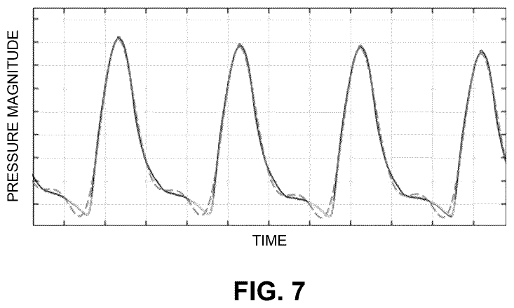

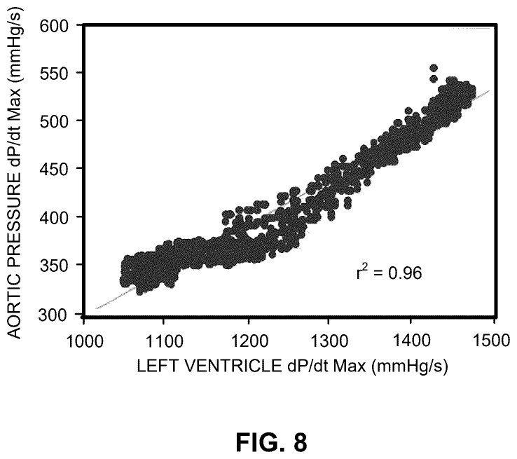

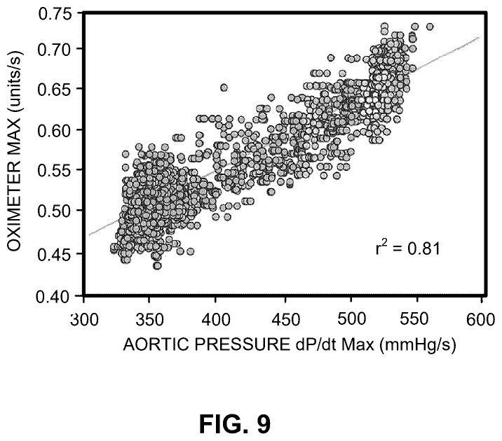

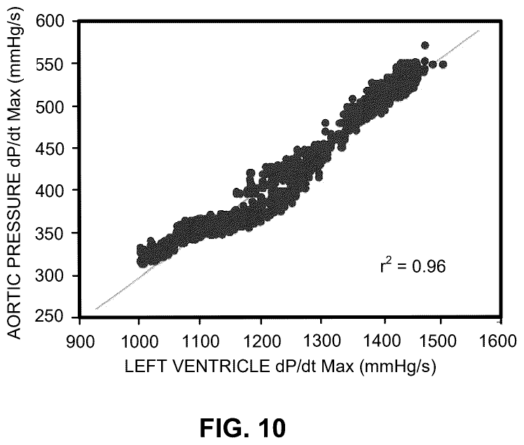

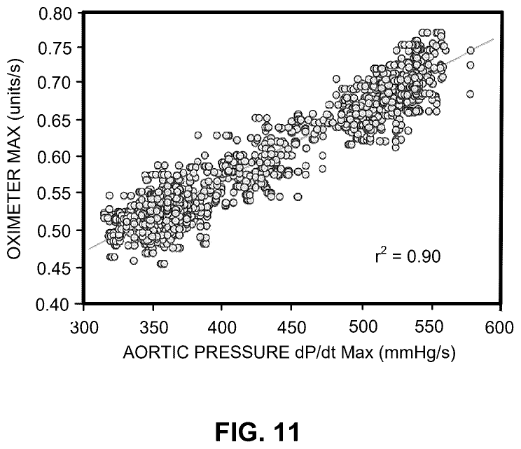

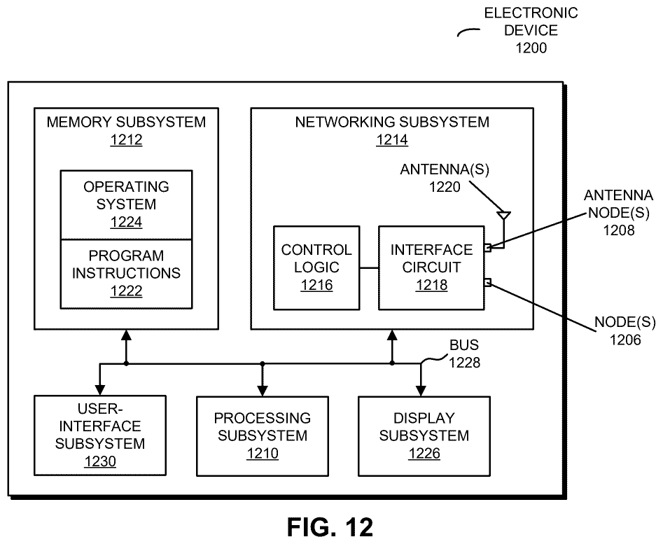

FIG. 9 is a drawing illustrating an example of maximum pulse oximeter values as a function of maximum pressure slope in the aorta in the first run of FIG. 7 in accordance with the present disclosure.

FIG. 10 is a drawing illustrating an example of maximum pressure slope in the aorta as a function of maximum pressure slope in the left ventricle in in a second run accordance with the present disclosure.

FIG. 11 is a drawing illustrating an example of maximum pulse oximeter values as a function of maximum pressure slope in the aorta in the second run of FIG. 9 in accordance with the present disclosure.

FIG. 12 is a block diagram illustrating an example of an electronic device in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

An electronic device that monitors blood pressure in vasculature of an individual is described. This electronic device performs the operations of: monitoring blood flow of the individual (e.g., using a pulse oximeter); calibrating the blood flow based at least in part on a predefined or predetermined comparison of the blood flow and blood pressure, e.g., in the individual's vasculature; and providing the calibrated blood flow of the individual as a function of time, where the calibrated blood flow indicates at least the blood pressure or a blood-pressure reading for the individual.

By monitoring the blood pressure using the calibrated blood flow, these monitoring techniques may assess heart function, as well as metrics such as heart rate and blood-flow volume. Moreover, the monitoring may be performed between instances of calibration of the monitoring, e.g., using a sphygmomanometer. Consequently, the monitoring may allow a non-invasive electronic device (such as a pulse oximeter) to be used, which may reduce the cost and facilitate continuous or more-continuous monitoring. Moreover, the monitoring may be less intrusive for the individual, thereby facilitating more-frequent or continuous monitoring. Note that the monitoring techniques may be used by one or more individuals (e.g., at home, in an outpatient setting, etc.) and/or by one or more medical professionals (e.g., in a doctor's office, a hospital, an intensive-care unit, during surgery, etc.). Furthermore, the monitoring techniques may enable or facilitate immediate (e.g., in less than 1 min) corrective or remedial action, such as: adjustment of another intervention (e.g., a medication) based at least in part on the calibrated blood flow provided by the monitoring techniques; and/or a recommendation to reposition one or more electrodes in an electrocardiogram (ECG) to an improved position or location on the individual. Additionally, the electronic device may be easier to use and may have a lower cost than existing approaches for cardiac monitoring (such as a Holter monitor, a cardiac monitor, e.g., a Zio patch from iRhythm Technologies, Inc., of San Francisco, California, an ECG, a sphygmomanometer, etc.). Consequently, the monitoring techniques may improve the quality and availability of the monitoring, may improve care of individuals and/or patients, and may reduce the cost of providing this care (e.g., by reducing in-patient evaluations and/or hospitalizations).

In the discussion that follows, electronic devices, computers and/or servers (which may be local or remotely located from each other) may communicate packets or frames in accordance with a wired communication protocol and/or a wireless communication protocol. The wireless communication protocol may include: a wireless communication protocol that is compatible with an Institute of Electrical and Electronics Engineers (IEEE) 802.11 standard (which is sometimes referred to as ‘Wi-Fi®,’ from the Wi-Fi Alliance of Austin, Texas), Bluetooth, Bluetooth low energy, a cellular-telephone network or data network communication protocol (such as a third generation or 3G communication protocol, a fourth generation or 4G communication protocol, e.g., Long Term Evolution or LTE (from the 3rd Generation Partnership Project of Sophia Antipolis, Valbonne, France), LTE Advanced or LTE-A, a fifth generation or 5G communication protocol, or other present or future developed advanced cellular communication protocol), and/or another type of wireless interface (such as another wireless-local-area-network interface). For example, an IEEE 802.11 standard may include one or more of: IEEE 802.11a, IEEE 802.11b, IEEE 802.11g, IEEE 802.11-2007, IEEE 802.11n, IEEE 802.11-2012, IEEE 802.11-2016, IEEE 802.11ac, IEEE 802.11ax, IEEE 802.11ba, IEEE 802.11be, or other present or future developed IEEE 802.11 technologies. Moreover, the wired communication protocol may include a wired communication protocol that is compatible with an IEEE 802.3 standard (which is sometimes referred to as ‘Ethernet’), e.g., an Ethernet II standard. However, a wide variety of communication protocols may be used. In the discussion that follows, Bluetooth and Ethernet are used as illustrative examples.

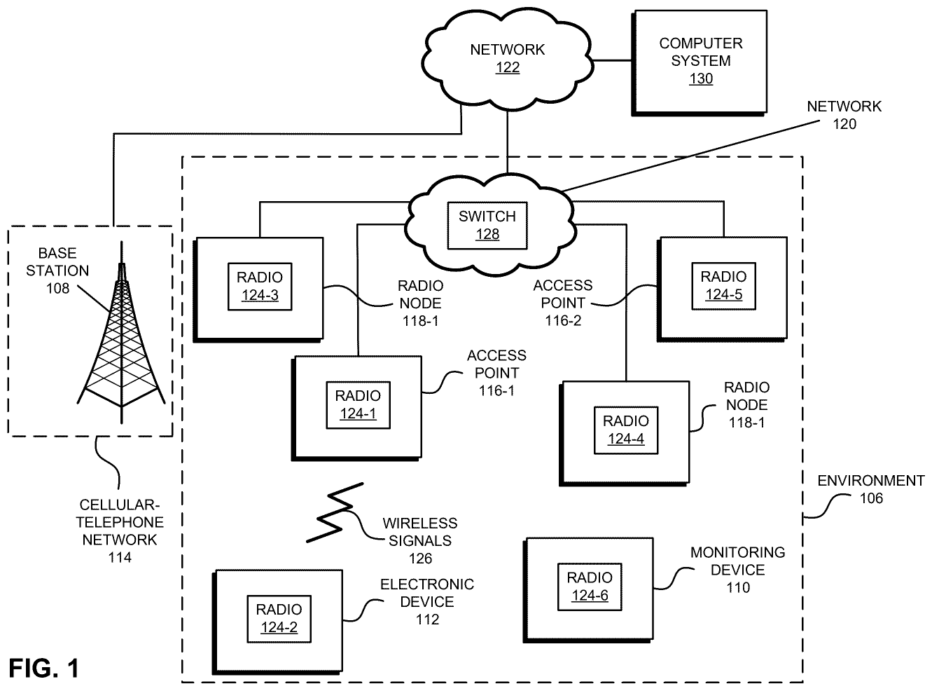

We now describe some embodiments of the monitoring techniques. FIG. 1 presents a block diagram illustrating an example of communication between a monitoring device 110 (which is sometimes referred to as an ‘electronic device’) and another electronic device 112 (such as a cellular telephone, a portable electronic device, or another type of electronic device, etc.) in accordance with an embodiment of the present disclosure. Moreover, electronic device 112 may optionally communicate via a cellular-telephone network 114 (which may include a base station 108), one or more access points 116 (which may communicate using Wi-Fi) in a wireless local area network (WLAN) and/or radio node 118 (which may communicate using LTE) in a small-scale network (such as a small cell). For example, radio node 118 may include: an Evolved Node B (eNodeB), a Universal Mobile Telecommunications System (UMTS) NodeB and radio network controller (RNC), a New Radio (NR) gNB or gNodeB (which communicates with a network with a cellular-telephone communication protocol that is other than LTE), etc. In the discussion that follows, an access point, a radio node or a base station are sometimes referred to generically as a ‘communication device.’ Moreover, one or more base stations (such as base station 108), access points 116, and/or radio node 118 may be included in one or more networks, such as: a WLAN, a small cell, a local area network (LAN) and/or a cellular-telephone network. In some embodiments, access points 116 may include a physical access point and/or a virtual access point that is implemented in software in an environment of an electronic device or a computer.

Furthermore, electronic device 112 may optionally communicate with computer system 130 (which may include one or more computers or servers, and which may be implemented locally or remotely to provide storage and/or analysis services) using a wired communication protocol (such as Ethernet) via network 120 and/or 122. Note that networks 120 and 122 may be the same or different networks. For example, networks 120 and/or 122 may be a LAN, an intra-net or the Internet. In some embodiments, the wired communication protocol may include a secured connection over transmission control protocol/Internet protocol (TCP/IP) using hypertext transfer protocol secure (HTTPS) with a JavaScript object notation (JSON) Web services connection. Additionally, in some embodiments, network 120 may include one or more routers and/or switches (such as switch 128).

In some embodiments, electronic device 112 and/or computer system 130 may optionally implement at least some of the operations in the monitoring techniques. Notably, as described further below, electronic device 112 and/or computer system 130 may optionally perform at least some of the analysis of measurement data acquired by monitoring device 110 (such as calibrating one or more blood-flow measurements), and may optionally provide feedback information to monitoring device 110 (such as providing the one or more calibrated blood-flow measurements).

As described further below with reference to FIG. 12, base station 108, monitoring device 110, electronic device 112, access points 116, radio node 118, switch 128 and/or computer system 130 may include subsystems, such as a networking subsystem, a memory subsystem and a processor subsystem. In addition, monitoring device 110, electronic device 112, access points 116 and radio node 118 may include radios 124 in the networking subsystems. More generally, monitoring device 110, electronic device 112, access points 116 and radio node 118 can include (or can be included within) any electronic devices with the networking subsystems that enable monitoring device 110, electronic device 112, access points 116 and radio node 118 to wirelessly communicate with one or more other electronic devices. This wireless communication can comprise transmitting access on wireless channels to enable electronic devices to make initial contact with or detect each other, followed by exchanging subsequent data/management frames (such as connection requests and responses) to establish a connection, configure security options, transmit and receive frames or packets via the connection, etc.

During the communication in FIG. 1, base station 108, monitoring device 110, electronic device 112, access points 116, radio node 118 and/or computer system 130 may wired or wirelessly communicate while: transmitting access requests and receiving access responses on wired or wireless channels, detecting one another by scanning wireless channels, establishing connections (for example, by transmitting connection requests and receiving connection responses), and/or transmitting and receiving frames or packets (which may include information as payloads).

As can be seen in FIG. 1, wireless signals 126 (represented by a jagged line) may be transmitted by radios 124 in, e.g., access points 116 and/or radio node 118 and monitoring device 110 or electronic device 112. For example, radio 124-1 in access point 116-1 may transmit information (such as one or more packets or frames) using wireless signals 126. These wireless signals are received by radio 124-2 in electronic device 112. This may allow access point 116-1 to communicate information to other access points 116 and/or electronic device 112. Note that wireless signals 126 may convey one or more packets or frames.

In the described embodiments, processing a packet or a frame in one or more electronic devices in monitoring device 110, electronic device 112, access points 116, radio node 118 and/or computer system 130 may include: receiving the wireless or electrical signals with the packet or the frame; decoding/extracting the packet or the frame from the received wireless or electrical signals to acquire the packet or the frame; and processing the packet or the frame to determine information contained in the payload of the packet or the frame.

Note that the wired and/or wireless communication in FIG. 1 may be characterized by a variety of performance metrics, such as: a data rate for successful communication (which is sometimes referred to as ‘throughput’), an error rate (such as a retry or resend rate), a mean-squared error of equalized signals relative to an equalization target, intersymbol interference, multipath interference, a signal-to-noise ratio, a width of an eye pattern, a ratio of number of bytes successfully communicated during a time interval (such as 1-10 s) to an estimated maximum number of bytes that can be communicated in the time interval (the latter of which is sometimes referred to as the ‘capacity’ of a communication channel or link), and/or a ratio of an actual data rate to an estimated data rate (which is sometimes referred to as ‘utilization’). While instances of radios 124 are shown in components in FIG. 1, one or more of these instances may be different from the other instances of radios 124.

In some embodiments, wireless communication between components in FIG. 1 uses one or more bands of frequencies, such as: 900 MHz, 2.4 GHz, 5 GHz, 6 GHz, 7 GHz, 60 GHz, the Citizens Broadband Radio Spectrum or CBRS (e.g., a frequency band near 3.5 GHz), and/or a band of frequencies used by LTE or another cellular-telephone communication protocol or a data communication protocol. Note that the communication between electronic devices may use multi-user transmission (such as orthogonal frequency division multiple access or OFDMA) and/or multiple-input multiple-output (MIMO).

Although we describe the network environment shown in FIG. 1 as an example, in alternative embodiments, different numbers or types of electronic devices may be present. For example, some embodiments comprise more or fewer electronic devices. As another example, in another embodiment, different electronic devices are transmitting and/or receiving packets or frames.

While FIG. 1 illustrates computer system 130 at a particular location, in other embodiments at least a portion of computer system 130 is implemented at more than one location. Thus, in some embodiments, computer system 130 is implemented in a centralized manner, while in other embodiments at least a portion of computer system 130 is implemented in a distributed manner.

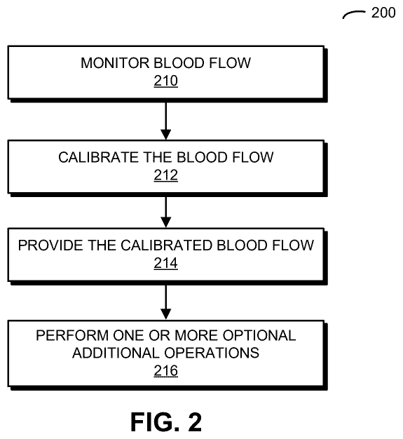

As described further below with reference to FIGS. 2-11, in order to address challenges associated with the regular or continuous use of a blood-pressure monitor (such as a sphygmomanometer), a monitoring device 110 (such as a pulse oximeter) may be remateably attached to or coupled to an individual. Monitoring device 110 may monitor blood pressure in vasculature of an individual. Notably, monitoring device 110 may monitor blood flow of the individual, such as by collecting or measuring one or more instances of pulse-oximetry waveforms. Then, monitoring device 110 may calibrate the blood flow based at least in part on a predefined or predetermined comparison of the blood flow and blood pressure, e.g., in the individual's vasculature; and provide the calibrated blood flow of the individual as a function of time, where the calibrated blood flow indicates at least the blood pressure or a blood-pressure reading for the individual.

Moreover, the monitoring of the blood flow may be performed continuously, periodically or as needed.

Furthermore, the providing may include displaying the calibrated blood flow of the individual.

Additionally, the calibrated blood flow may be sinusoidal, with each pulse in the calibrated blood flow having a peak amplitude A. The calibrated blood flow may also have a time interval Δt between the peak amplitude of a given pulse and the peak amplitude of the immediately preceding pulse. Thus, the calibrated blood flow may include a heart rate corresponding to Δt and/or a blood-flow volume corresponding to Δt and A. Consequently, in some embodiments, monitoring device 110 may: compute the heart rate and/or the blood-flow volume based at least in part on the calibrated blood flow; and/or may provide the computed heart rate and/or blood-flow volume.

In some embodiments, monitoring device 110 may perform a calibration, where the calibration includes measuring an instance of the comparison of the blood flow and the blood pressure using, at least in part, a sphygmomanometer or another type of blood-pressure measurement device. For example, the instance of the comparison may be measured at a steady-state condition for the relationship between the individual's blood-flow measurement A and their blood-pressure reading Pmeasured. Note that Pmeasured may be a difference between a systolic pressure and a diastolic pressure. This steady-state condition may then be used by monitoring device 110 to perform the calibration (or to compute the calibrated blood flow, Acalibrated). During the comparison, monitoring device 110 may concurrently obtain a measurement of the blood flow and the blood pressure, e.g., in the steady-state condition. In some embodiments, the predefined or determined comparison of the blood flow and the blood pressure may correlate or include changes in the blood flow ±ΔA with changes in the blood pressure ±ΔP.

Note that, for a constant blood-flow condition, the predefined or predetermined comparison may be expressed as A=P/R, where R is a factor representing the individual's vascular resistance to blood flow. In this ratio relationship, changes in blood flow ±ΔA and changes in blood pressure ±ΔP may be equated to each other for correlation purposes as ±ΔA/Acalibrated ≈±ΔP/Pmeasured. The correlation of blood flow with blood pressure may be made relative to a previously established steady-state condition. For this purpose, the steady-state condition may be expressed as (ΔP)base=Psystolic−Pdiastolic.

Consequently, when providing the calibrated blood flow, monitoring device 110 may display blood-pressure variations ΔP corresponding to variations in the blood flow ±ΔA. As noted previously, this correspondence may be made based at least in part on the predefined or predetermined comparison of the blood flow and the blood pressure having a constant (ΔP)base. For the purpose of assessing blood flow in the individual's vasculature, monitoring device 110 may indicate whether there are any consequent changes in heart rate ±Δt that are associated with concurrently measured ±ΔA. Notably, monitoring device 110 may selectively present ±ΔP in the context of either a first operational state wherein Δt is constant, or a second operational state in which Δt is variable.

In the first operational state, Δt may be constant and, to maintain a proper constant blood-flow relationship, R may be varied whenever A is varied in the predefined or predetermined comparison A=P/R. Notably, with a +ΔA there may be a comparable change in R >1, and with a −ΔA there may be a comparable change in R <1. In the short term (such as a few seconds or a few minutes), this relationship may be considered valid because R is anatomically slow to vary. On the other hand, R may become a factor over a relatively longer term.

In the second operational state, Δt may be variable, and R may remain constant to maintain the operational relationship between ±ΔA and ±ΔP. Nonetheless, ±ΔA and ±ΔP may vary somewhat. In this latter case, recalibration (e.g., using a sphygmomanometer) may be needed. Thus, in each operational state, the predefined or predetermined comparison may determine a blood pressure P (e.g., for display) that may include clinical information pertinent to the individual's condition.

In some embodiments, monitoring device 110 (or an associated monitor or display) may record variations of ±ΔA and ±Δt in the blood flow during a predefined or predetermined time duration. These measurements may be compared with previous measurements to determine whether ±ΔA and ±Δt have sufficiently stabilized during the predefined or predetermined time duration to identify a new value for the blood flow (e.g., A′). If so, the steady-state condition may be recalibrated. As noted previously, this calibration may be performed (e.g., periodically) using a sphygmomanometer to obtain new blood-pressure readings P′measured to recalibrate a new value for the blood flow A′ as A′calibrated for use with P′measured to identify a new steady-state condition for the individual.

In these ways, the monitoring techniques may facilitate dynamic and real-time monitoring of cardiac pumping function of the individual. Because the monitoring is non-invasive, the monitoring techniques may be easier to use and may have a lower cost than existing approaches for blood-pressure monitoring. Moreover, the monitoring techniques may enable or facilitate immediate (e.g., less than 1 min) corrective or remedial action. Consequently, the monitoring techniques may improve the quality and availability of assessments of overall heart function, may improve care of individuals and/or patients, and may reduce the cost of providing this care (e.g., by reducing in-patient evaluations and/or hospitalizations).

We now describe embodiments of the method. FIG. 2 presents a flow diagram illustrating an example of a method 200 for providing calibrated blood flow of an individual. This method may be performed by an electronic device, such as monitoring device 110.

During operation, the electronic device may monitor blood flow (operation 210) of the individual. Then, the electronic device may calibrate the blood flow (operation 212) based at least in part on a predefined or predetermined comparison of the blood flow and blood pressure, e.g., in the individual's vasculature. Next, the electronic device may provide the calibrated blood flow (operation 214) of the individual as a function of time, where the calibrated blood flow indicates at least the blood pressure or a blood-pressure reading for the individual.

Note that, during the monitoring (operation 210), the electronic device may be coupled or attached to the individual, such as a patient.

Moreover, the monitoring of the blood flow (operation 210) may be performed continuously, periodically or as needed.

Furthermore, the monitoring of the blood flow (operation 210) may be performed using a pulse oximeter. For example, during the monitoring (operation 210), the electronic device may acquire an instance of a pulse oximeter waveform of the individual.

Additionally, the providing (operation 214) may include displaying the calibrated blood flow of the individual.

In some embodiments, the calibrated blood flow may be sinusoidal, with each pulse in the calibrated blood flow having a peak amplitude A. The calibrated blood flow may also have a time interval Δt between the peak amplitude of a given pulse and the peak amplitude of the immediately preceding pulse. Thus, the calibrated blood flow may include a heart rate corresponding to Δt and/or a blood-flow volume corresponding to Δt and A. Consequently, in some embodiments, the electronic device may perform one or more optional additional operations (operation 216), such as: computing the heart rate and/or the blood-flow volume based at least in part on the calibrated blood flow; and/or providing the computed heart rate and/or blood-flow volume.

Moreover, the electronic device may perform a calibration, where the calibration includes measuring an instance of the comparison of the blood flow and the blood pressure using, at least in part, a sphygmomanometer or another type of blood-pressure measurement device. For example, the instance of the comparison may be measured at a steady-state condition for the relationship between the individual's blood-flow measurement A and their blood-pressure reading Pmeasured. Note that Pmeasured may be a difference between a systolic pressure and a diastolic pressure. This steady-state condition may then be used by the electronic device to perform the calibration (or to compute the calibrated blood flow, Acalibrated). During the comparison, the electronic device may concurrently obtain a measurement of the blood flow and the blood pressure, e.g., in the steady-state condition. In some embodiments, the predefined or determined comparison of the blood flow and the blood pressure may correlate or include changes in the blood flow ±ΔA with changes in the blood pressure ±ΔP.

Note that, for a constant blood-flow condition, the predefined or predetermined comparison may be expressed as A=P/R, where R is a factor representing the individual's vascular resistance to blood flow. In this ratio relationship, changes in blood flow ±ΔA and changes in blood pressure ±ΔP may be equated to each other for correlation purposes as ±ΔA/Acalibrated ≈±ΔP/Pmeasured. The correlation of blood flow with blood pressure may be made relative to a previously established steady-state condition. For this purpose, the steady-state condition may be expressed as (ΔP)base=Psystolic−Pdiastolic.

Consequently, when providing the calibrated blood flow (operation 214), the electronic device may display blood-pressure variations ±ΔP corresponding to variations in the blood flow ±ΔA. As noted previously, this correspondence may be made based at least in part on the predefined or predetermined comparison of the blood flow and the blood pressure having a constant (ΔP)base. For the purpose of assessing blood flow in the individual's vasculature, the electronic device may indicate whether there are any consequent changes in heart rate Δt that are associated with concurrently measured ±ΔA. Notably, the electronic device may selectively present ±ΔP in the context of either a first operational state wherein Δt is constant, or a second operational state in which Δt is variable.

In the first operational state, Δt may be constant and, to maintain a proper constant blood-flow relationship, R may be varied whenever A is varied in the predefined or predetermined comparison A=P/R. Notably, with a +ΔA there may be a comparable change in R >1, and with a −ΔA there may be a comparable change in R <1. In the short term (such as a few seconds or a few minutes), this relationship may be considered valid because R is anatomically slow to vary. On the other hand, R may become a factor over a relatively longer term.

In the second operational state, Δt may be variable, and R may remain constant to maintain the operational relationship between ±ΔA and ±ΔP. Nonetheless, ±ΔA and ±ΔP may vary somewhat. In this latter case, recalibration may be needed. Thus, in each operational state, the predefined or predetermined comparison may determine a blood pressure P (e.g., for display) that may include clinical information pertinent to the individual's condition.

In some embodiments, the electronic device (or an associated monitor or display) may record variations of ±ΔA and ±Δt in the blood flow during a predefined or predetermined time duration. These measurements may be compared with previous measurements to determine whether ±ΔA and ±Δt have sufficiently stabilized during the predefined or predetermined time duration to identify a new value for the blood flow (e.g., A′). If so, the steady-state condition may be recalibrated. As noted previously, this calibration may be performed (e.g., periodically) using a sphygmomanometer to obtain new blood-pressure readings P′measured to recalibrate a new value for the blood flow A′ as A′calibrated for use with P′measured to identify a new steady-state condition for the individual.

In some embodiments of method 200, there may be additional or fewer operations. Furthermore, the order of the operations may be changed, and/or two or more operations may be combined into a single operation.

For example, in some embodiments, at least some of the aforementioned operations may be performed by a computer system (which may include at least a computer), which may be coupled to the electronic device. For example, the electronic device may provide, to the computer system, the monitored blood flow. In response, the computer system may compute the calibrated blood flow, which is then provided to the electronic device. Thus, the computer system may perform at least sone of the aforementioned operations.

We now further describe the monitoring techniques. A consequence of the interrelationship between the blood-flow waveform and blood pressure is that there may be three separately measurable characteristics of particular importance. These characteristics are variable and include: the peak amplitude A of a pulse in the blood-flow waveform; the time interval Δt between these peak amplitudes (which is sometimes referred to as the ‘heart rate’); and blood pressure P. When considered together, collectively, these variables can lead to clinical conclusions that otherwise may have been missed.

With this in mind, the disclosed monitoring techniques may provide a pulse oximeter that can be (e.g., periodically) calibrated using a sphygmomanometer to provide (e.g., simultaneously) blood-pressure readings with blood-flow waveform information. This information may be provided continuously, periodically or as needed. In some embodiments, the monitoring techniques may incorporate heart-rate information (±Δt) together with blood-flow data (±ΔA) and blood-pressure measurements (±ΔP) to provide more-comprehensive information (which may be displayed) for clinical personnel with which to assess a patient's condition. Moreover, in some embodiments, the monitoring technique may allow noninvasive blood-pressure monitoring to elucidate new data concerning normal and abnormal states to greatly increase the diagnostic power and patient safety in the clinical environment. Furthermore, in some embodiments, the monitoring technique may provide a system for monitoring a patient's clinical condition (e.g., continuously) that is easy to install, is simple to operate, and which is comparatively cost effective.

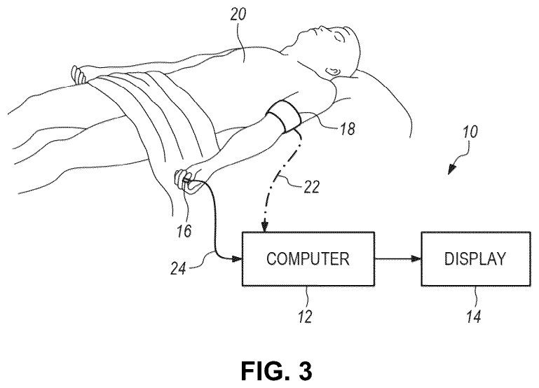

FIG. 3 presents a drawing illustrating an example of components of a system 10 connected to a patient in a clinical environment for taking a blood pressure reading. As shown in FIG. 3, system 10 may include a computer 12, which may be connected to a display 14. A pulse oximeter 16 and a sphygmomanometer cuff 18 may typically be placed on a patient 20. In FIG. 3, the dot-dash line 22 connecting sphygmomanometer cuff 18 with computer 12 is shown differentiated to thereby indicate that this connection is not continuous. Instead, as is disclosed below, sphygmomanometer cuff 18 may be used primarily for the purpose of periodically calibrating pulse oximeter 16. On the other hand, the solid line 24 is shown in FIG. 3 to indicate a permanent connection between pulse oximeter 16 and patient 20.

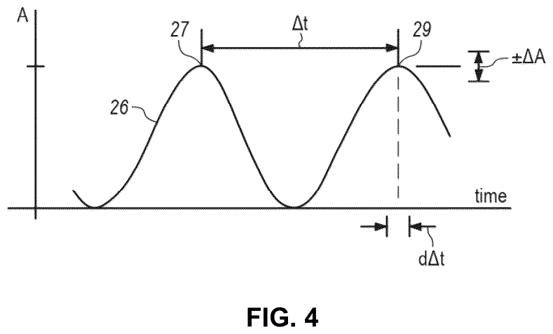

The purposes of pulse oximeter 16 and sphygmomanometer cuff 18 may be, respectively, of types well known in the industry. Notably, sphygmomanometer cuff 18 may be used to take periodic blood pressure readings, P, from patient 20 that will include both a systolic pressure, Psystolic, and a diastolic pressure, Pdiastolic. On the other hand, pulse oximeter 16 may be used to identify a blood-flow waveform 26. This is shown in FIG. 4, which presents a drawing illustrating an example of a blood-flow waveform of a patient.

In FIG. 4, it is to be appreciated that blood-flow waveforms 26 may be essentially sinusoidal and they may have certain characteristics in common. Of these, the characteristics of particular interest for the disclosed monitoring techniques may include changes in the magnitude ΔA of successive peaks in waveform 26 and the time duration Δt between successive peaks. FIG. 4 shows these variable characteristics with reference to peaks 27 and 29. Note that both variables (ΔA and Δt) may be measured by pulse oximeter 16.

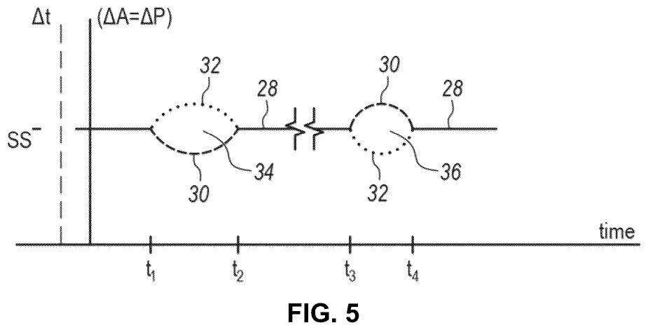

A consequence of changes in the respective magnitudes of ΔA and Δt is that as one increases the other typically decreases. As discussed previously, for purposes of the disclosed monitoring techniques, a steady state may be established when patient 20 is resting. With patient 20 resting, a base relationship may be created where A=P/R is constant, and R equals 1. In this base relationship (ΔA/sec)base=(ΔP)base=0. Nevertheless, when patient 20 has a short-term episode with a ±ΔA, the relationship ΔA=ΔP may still be considered acceptable, at least in the short term. The consequence of this may be best appreciated with reference to FIG. 5, which presents a drawing illustrating an example of a graphical presentation of a tracing profile for the patient's blood flow.

Examples for operation of the disclosed monitoring techniques are provided below. In these examples, the steady-state case is presented when R, in the expression A=P/R, is constant, e.g., with R=1. This situation may be such that (ΔA/Δt)base ≈ΔP/R >>±ΔP. Also, it may be appreciated that (ΔP)base may be determined on a case-by-case basis and may vary accordingly.

By way of example, consider an operational model being established where (ΔP)base=ΔPsystolic−ΔPdiastolic=120 mmHg−60 mmHg=60 mmHg, where ΔPsystolic is approximated at ⅘ΔP, and ΔPdiastolic is approximated at ⅕ΔP. Moreover, note that, when there is an increase in Δt, there will be a drop in P. And vice-versa: when there is a decrease in Δt, there will be a rise in P.

### Example 1 (Increase in Heart Rate Δt)

In accordance with the expression A=P/R, for an increase in heart rate by a factor of 2 (e.g., Δt decreases by ½), R in the expression A=P/R may also be considered equal to 2. Thus, ΔP=(ΔP)base/2=30 for a decrease in ΔP. Thus, for this example, (ΔP)display may equal [120−4/5·(30)]/[60−1/5·(30)]=96/54.

### Example 2 (Decrease in Heart Rate Δt)

In accordance with the expression A=P/R, for a decrease in heart rate by a factor of 15% (e.g., Δt increases by a factor of 1/0.85=1.18), R in the expression A=P/R may also be considered equal to 0.85. Thus, ΔP=(ΔP)base/0.85=60/0.85=70.1 for an increase in ΔP. For this example, (ΔP)display may equal [120+⅘·(70.1)]/[60+⅕·(70.1)]=176/74.

In FIG. 5, a tracing profile 28 is shown for waveform 26. As indicated in FIG. 5, tracing profile 28 may have two different characteristic components, Δt and ΔA. These characteristic components are individually shown respectively in FIG. 5 as a dashed line 30 for Δt, and a dotted line 32 for ΔA. In a steady-state (SS) condition for waveform 26, e.g., when ΔA/Δt is considered to be constant, tracing profile 28 is shown by a solid line. FIG. 5 also shows that in keeping with the expression A=P/R, ΔA ≈ΔP when these variables are in the steady state, SS.

Moreover, in FIG. 5, examples of two different episodes of clinical interest for the monitoring techniques are shown. One is an increased pressure episode 34 for patient 20, and the other is a decreased pressure episode 36 for patient 20. During increased pressure episode 34, Δt decreases (corresponding to a heart-rate increase) as indicated by dashed line 30. Therefore, display 14 may show an increase in ΔP. On the other hand, during decreased pressure episode 36, Δt may increase (corresponding to a heart-rate decrease) and ΔP may decrease. As recognized by the disclosed monitoring techniques, Δt and ΔA (or ΔP) may both be measured by pulse oximeter 16.

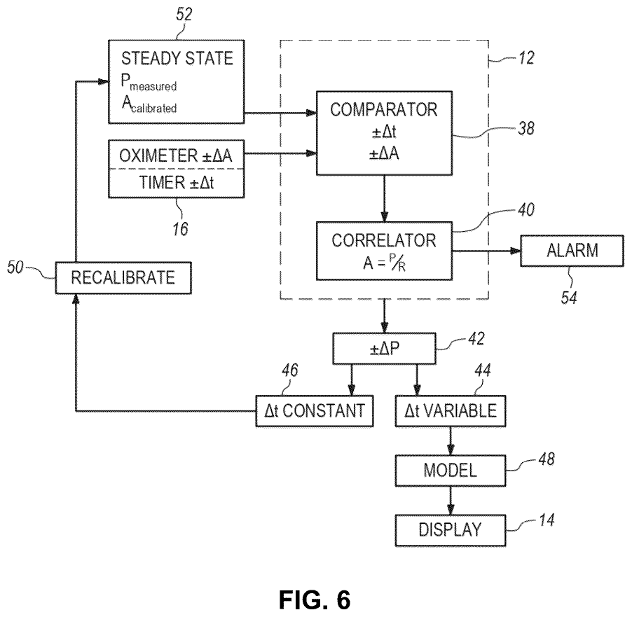

As shown in FIG. 6, which presents a drawing illustrating an example of the interactive operation of components in the system, computer 12 may include a comparator 38 and a correlator 40. Comparator 38 may receive information from pulse oximeter 16 regarding both ±ΔA and ±Δt. These variables may then be correlated by correlator 40 to determine an output ±ΔP that is analyzed as indicated by action block 42. Specifically, ±ΔP may be selectively analyzed relative to either a variable Δt at action block 44, or relative to a constant Δt (i.e., Δt=0) at action block 46.

When Δt is variable (block 44), model 48 for Pbase may be evaluated as discussed previously. As was also discussed previously, the result of this evaluation may be shown on display 14 (and, more generally, may be provided to a user and/or stored in memory). On the other hand, when Δt is considered a constant (block 46), the variable R may be evaluated for possible recalibration (block 50) of the expression A=P/R or for sounding an alarm 54. In the event that recalibration is implemented, the steady state SS for Pmeasured and Acalibrated with a new (ΔP)base may be established using pulse oximeter 16 and sphygmomanometer 18. This action is indicated by action block 52.

While the particular system and method for correlating pulse-oximetry waveform signals with blood pressure have been described, these embodiments are illustrations of the monitoring techniques. In other embodiments, there may be additional or fewer components or features, two or more components or features may be combined, and/or one or more components or features may be separated or divided. Thus, in some embodiments, the operations in the monitoring techniques may be performed by a single electronic device, instead of a separate pulse oximeter 16, computer 12 and display 14 shown in FIG. 3. Alternatively, the monitoring techniques may be performed by an electronic device and a display. Consequently, the monitoring techniques may be implemented in a distributed or a centralized manner.

In some embodiments, at least some of the operations in the monitoring techniques may be performed using a pretrained analysis model, which may include the predefined or predetermined comparison of the blood flow and the blood pressure. For example, the pretrained analysis model may include a neural network. More generally, a variety of supervised-learning techniques may be used to generate the pretrained model. For example, the supervised-learning techniques may include: a linear model, a nonlinear model, Support Vector Machines, LASSO, etc.

We now describe calibration of a pulse-oximetry waveform. FIG. 7 presents a drawing illustrating an example of calibration of a pulse-oximetry waveform in accordance with the present disclosure. The pulse-oximetry waveform is very similar to that recorded from the aorta or any large artery in the human body. Note that time is on the horizontal axis and pressure magnitude is on the vertical axis.

The raw red and infra-red optical signals from the pulse oximeter may have a similar configuration. These blood-flow signals may be superimposed on the simultaneously obtained blood-pressure signals to make the transition from blood flow to blood-pressure calibration. This relationship may continue to be a direct one as long as the blood flow remains constant and the vascular resistance is unchanged (which is sometimes referred to as ‘Ohms Law for Physiologists’). Under these conditions, blood flow equals blood pressure and, thus, blood pressure can be continuously monitored via the blood flow.

The blood-flow signal may also be calibrated by obtaining systolic and diastolic blood pressure with a sphygmomanometer while recording the blood-flow signal. The peak waveform pressure would be the systolic pressure obtained with the sphygmomanometer, while the diastolic pressure would be at the portion at the last third of the waveform where there is a marked change in slope (the waveform becomes more horizontal).

We now further describe the monitoring techniques. For healthcare situations, there are many possible reasons why there is a need to measure and monitor an individual's blood pressure. Typically, this is done by intermittently using a sphygmomanometer. Although a sphygmomanometer is a useful and reliable medical device for measuring an individual's blood pressure, its repetitive use may not be practical when ongoing (e.g., continuous) monitoring of an individual's blood pressure for extended time durations is necessary or preferable.

Moreover, a pulse oximeter is capable of continuously measuring blood flow. Notably, pulse oximeters may provide measurements of blood oxygen saturation levels that are indicative of the volume of blood being measured. Pulse-oximeter measurements, however, like blood-pressure measurements, are pulsatile. The respective pulses, however, have different dimensional characteristics. In the context of the monitoring techniques, the similarities and differences in the relationship between blood flow and blood pressure are important for several reasons. The similarities include:
Blood flow is a direct temporal consequence of blood-pressure variations produced during a heart muscle cycle (or pulse); and
The dimensional characteristics of blood flow and blood pressure have concurrence in that blood pressure affects blood flow repetitively during each heart muscle cycle. On the other hand, for measurement purposes, there are distinctive differences between blood pressure and blood flow that may need to be reconciled. These differences include:
The maximum amplitude of a blood-pressure measurement, Pmax, and the maximum amplitude of a flood-flow measurement, Fmax, during a heart-muscle cycle are inversely proportional;
Pmax and Fmax occur at different times during a heart-muscle cycle;
The respective rates of change for ΔPmax and ΔFmax from pulse-to-pulse may be different, or ΔPmax may not equal ΔFmax; and
A sphygmomanometer does not measure both systolic and diastolic pressures during the same heart muscle cycle.

For these reasons, the monitoring techniques may recalibrate (e.g., continuously) blood-pressure measurements with corresponding blood-flow measurements. This may be done so that a pulse oximeter can be used alone to monitor blood-pressure trends for successive heart muscle functions over a predetermined time duration. Moreover, the monitoring techniques may include the use of a line graph (or relationship) in an electronic device, which can be used to calibrate blood-pressure trends with a pulse oximeter. Furthermore, the monitoring techniques may provide an electronic device that measures blood pressure using a pulse oximeter that is easy to manufacture, is simple to use, and/or is cost-effective.

In some embodiments, the monitoring techniques may use blood-flow measurements from an individual as indications of the individual's blood pressure trends. For example, the monitoring techniques may be performed using: a sphygmomanometer, a pulse oximeter, and a collator. (Some or all of these components may be integrated, in hardware and/or software, in one or more devices.) The sphygmomanometer may be used to periodically measure an individual's blood pressure P. On the other hand, the pulse oximeter may be used to measure the individual's blood-oxygen saturation levels as indicators of his/her blood flow F. This monitoring may be performed continuously. The collator may then collect the blood-pressure measurements and the blood-flow measurements and combine selected dimensional aspects of these measurements into datasets.

For each dataset, the sphygmomanometer may measure a maximum blood-pressure measurement Psystolic near the beginning of each heart-muscle cycle. The sphygmomanometer may also measure a Pdiastolic during the same heart-muscle cycle. In a separate operation, the pulse oximeter may measure blood-flow variations that include a maximum amplitude Fmax near the end of each heart-muscle cycle. Furthermore, the sphygmomanometer may also be used to measure a heart pulse rate from an individual to establish the duration for a heart-muscle cycle.

Note that P (blood pressure) and Fmax (blood flow) may have an inverse relationship that may be considered in the context of a heart-muscle cycle. In order to account for this fact, the collator may collect Psystolic, Pdiastolic and Fmax measurements during each heart-muscle cycle. From these measurements, Psystolic and Pdiastolic may be combined to establish a dataset that can then be used as a steady-state quantified Δss. Δss may equal Psystolic−Pdiastolic and it may be constant. Although, blood pressure changes, ΔPsystolic and Pdiastolic may not be directly equal to the blood-flow changes ΔFmax, the quantified Δss for Psystolic and Pdiastolic for blood pressures may be considered concurrent with ΔFmax.

It happens that, in a sequence of heart-muscle cycles, from one heart-muscle cycle to the next, Pdiastolic may be more reliable for use as a reference point than Psystolic. Thus, for an operation of the monitoring techniques, Pdiastolic values may be used as reference points along a line graph (or relationship) for a sequence of respective Δss values. Functionally, the resultant line graph may then be used for an extended time period to calibrate Fmax measurement from the pulse oximeter with a blood pressure P from Δss.

In detail, a line graph may be created for the monitoring techniques using at least two reference points. Each reference point may be separately created with a Psystolic and a Pdiastolic measurement, which may be combined in a dataset for use as a quantified Δss at a location on the line graph. Furthermore, each quantified Δss may be individually established when the individual is posed in different positions. Consequently, the quantified Δss values may be used to create the line graph, with each location along the line graph providing a unique comparison Δss. Thus, the line graph may be used to calibrate an F measurement from the pulse oximeter with a correlated P. With this calibration, the result is that the correlated P (which is sometimes referred to as a calibrated blood flow) may be shown on a display as an indication of blood pressure.

As noted previously, several factors may be considered during the creation of a line graph. For example, Pmax and Fmax may be measured separately, and they may have an inverse relationship. Furthermore, between different quantified Δss values, the rate of change ΔPmax may not equal to the rate of change in ΔFmax. Thus, each unique steady-state comparison Δss along the line graph may change and may have a new value that accounts for the fact that in a sequence each Δss=(P ±ΔP) and (F ±ΔF).

A methodology for the monitoring techniques may require a sequence of operations to obtain blood-flow measurements from an individual, which may be continuously monitored and used as real-time indications of the individual's blood pressure. Furthermore, the methodology may provide instructions that are useful for manufacturing an electronic device in accordance with the monitoring techniques. It is also useful for subsequently monitoring an individual's blood pressure with the electronic device.

In use, a sphygmomanometer may be positioned on an individual to measure his/her blood pressure P. At the same time, a pulse oximeter may also be positioned on the individual to measure blood flow F. With this oximeter/sphygmomanometer combination, a maximum blood pressure Pmax may be measured by the sphygmomanometer, and a contemporary maximum blood flow Fmax may be measured by the pulse oximeter. A pulse-rate measurement may also be obtained from the sphygmomanometer and be used to determine the time duration for the individual's heart-muscle cycle.

Because Pmax and Fmax may have concurrence in the same heart-muscle cycle, the measured values for Pmax and Fmax may be collated together as components for use as a same dataset. Each collated dataset may thereby be combined into a steady-state quantified comparison Δss. Note that each quantified comparison Δss may be unique with blood-pressure and blood-flow measurements. More specifically, each quantified comparison Δss may include measurements that are taken from the individual while he/she is posed in different positions, such as standing, sitting, or lying down.

A line graph for the monitoring techniques may be created using the Pmax and Fmax values taken for successive quantified comparisons Δss. Notably, F may establish the horizontal axis of the line graph, while P may establish the vertical axis. Because Pmax and Fmax may have an inverse relationship, the horizontal axis of the line graph may show a decreasing value for F. On the other hand, the vertical axis of the line graph may show an increasing value for P. With this inverse relationship, each location on the resulting line graph, between quantified comparisons Δss, may represent a specific comparison Δss having unique values for P and F.

Note that between any two quantified comparisons Δss, at each location on the line graph, the rate of change ΔP may not equal to the rate of change in ΔF. Consequently, they may need to be considered separately for each successive comparison Δss. Therefore, values for a successive Δss, using values from its predecessor Δss, may equal (P±ΔP) and (F ±ΔF). When using a line graph as disclosed in the monitoring techniques, values for F, which may be continuously measured by a pulse oximeter, may be directly correlated at every location along the line graph with a corresponding P from the same comparison Δss.

In some embodiments, a quantified comparison Δss may be periodically recalibrated with updated Pmax measurements taken by the sphygmomanometer (e.g. every 30 minutes). Furthermore, depending on the number of multiple quantified comparisons Δss that are measured, they may all be collectively used as different reference points to create a continuous line graph with differently oriented line segments (e.g., a 3-point line graph). For example, a 3-point line graph may be created having two different line segments. In this case, each line segment may be established between only two different quantified comparisons Δss.

In some embodiments, a system may use blood-flow measurements F from an individual as indications of the individual's blood pressure P. This system may include an electronic device that is coupled or connected directly with the individual. Notably, a sphygmomanometer may be fitted onto the individual to obtain blood-pressure measurements P from the individual. Similarly, a pulse oximeter may be fitted onto the individual to obtain blood-flow measurements F from the individual. Both measurements, P and F may be taken during a same quantified comparison Δss.

Moreover, the sphygmomanometer may be is connected directly with the collator of the electronic device. Note that blood-pressure measurements P may be taken intermittently. The minor interruptions between P measurements may be primarily due to time limitations and the labor-intensive nature for operation of the sphygmomanometer. On the other hand, blood-flow measurements F from the pulse oximeter may be taken continuously with minimal, if any, interruption. In this operation, it is to be appreciated that the collator may function to collect blood pressure measurements P along with blood-flow measurements F (e.g., in an automated or semi-automated manner). During this operation, the collator may also function to then combine the P and F measurements for comparison purposes.

In the monitoring techniques, the combination of a single P measurement and a single F measurement may constitute a dataset that is then quantified. Notably, quantification of the dataset may include creation of a steady-state quantified comparison Δss for the measurements. Note that multiple quantified comparisons Δss may be used during operation of the system.

The data used to establish a quantified comparison Δss may include Psystolic and Pdiastolic, and the value for Fmax in a blood-flow data trace. For the system, a common nexus between Pdiastolic and Fmax may last for the same time duration Δt, which may be the time required for a sphygmomanometer to record Δss. Notably, Δt may be the time required for a sphygmomanometer 16 to measure at least one Pdiastolic in a sequence of heart-muscle cycles. In some embodiments, Pdiastolic may be more reliable than Psystolic as a reference for identifying P over extended time periods.

In a blood-flow trace, an episode may be selected from within a longer episode in a blood-pressure trace. Notably, a measured Pdiastolic and a single Fmax may occur together only once during Δt of the longer episode. On the other hand, occurrences of Fmax may occur continuously for each heart-muscle cycle during Δt.

In detail it happens that Δt may typically extend through several heart-muscle cycles. The consequence here is that because of the operational requirements of a sphygmomanometer, the time interval between the Pdiastolic measured in one heart cycle and Pdiastolic that can be measured for the next heart-muscle cycle may necessarily be delayed by Δt. Although Δt may last for a few heart-muscle cycles, there is only one Pdiastolic that can be measured during the longer episode.

Moreover, it happens that, during any longer episode for the sphygmomanometer, there may be several separate episodes that occur sequentially for the pulse oximeter. Within the time duration Δtox of each episode for the pulse oximeter there may always be both a Pmax and an Fmax.

Although only one Pdiastolic can be measured somewhere within the time duration Δt, both this Pmax and an Fmax may occur at least once in the same longer episode during Δt. Thus, for purposes of the system, the measurements of Pmax and Fmax may be effectively considered to be concurrent. Accordingly, they can be used as components for establishing a quantified Δss.

During a quantified Δss that may be blood-pressure variations for P, and blood-flow variations for F. Note that the value of P variations may increase in an upward direction. At the same time, the value of F variations may increase in a downward direction. This may happen because, with an increased volume of blood flow F, light absorption also increases. However, with increased light absorption, the magnitude of light signals measured by a pulse oximeter are decreased. Thus, the inverse relationship. A compensation for this inverse relationship by the collator, which may use any F and may only measure Pdiastolic during a longer episode, is sometimes referred to as a quantified comparison Δss. In the monitoring techniques, quantified comparisons Δss may be used to create a line graph.

Furthermore, a line graph may constitute a continuous sequence of comparisons Δss. Notably, the line graph may be established between quantified comparisons Δss which are respectively located at different reference points. All locations along the line graph, as well as locations on extensions beyond the end points, may each identify a unique F and P relationship for a unique Δss. For example, consider a measured value for F from the pulse oximeter at a point. This point may reference a point on the graph line that calibrates F to a value for P. It is this value for P that corresponds with a unique Δss that is observed by an individual as his/her blood pressure.

We now describe recorded observations of a sequence of events following coronary artery occlusion to cardiac muscle downstream. These observations provide an assessment of the earliest and most sensitive measures of function and malfunction. Notably, pig hearts were instrumented with ultrasonic crystals so that small regions of contracting muscle or shortening could be monitored, blood pressure and ECGs were simultaneously recorded. The artery subtending the area where shortening was being monitored was suddenly occluded and within about 5 s shortening began to be progressively diminished. ECG changes indicating this event did not occur until about one minute after the occlusion. Changes in blood pressure did not occur early on. The conclusion was that regional contractility was the most sensitive measure of regional perfusion and performance. Global ejection fraction was not measured in these experiments, but other work suggests it would lag behind these regional changes in shortening. However, dP/dT max for left ventricular pressure would predictably be changing early reflecting these regional changes.

Moreover, in some embodiments of the measurements on pigs, two runs were captured in which pressures were measured simultaneously in the left ventricle, the aorta and the pulse oximeter reading from the tongue. FIGS. 8 and 10 are drawings illustrating examples of maximum pressure slope in the aorta as a function of maximum pressure slope in the left ventricle in in a first run or a second run. Moreover, FIGS. 9 and 11 are drawings illustrating an example of maximum pulse oximeter values as a function of maximum pressure slope in the aorta in the first run of FIG. 8 or the second run of FIG. 10. Notably, FIGS. 8-11 shown simultaneous matched beats plotted for the maximum rate of rise of the waveform from left ventricle (LV), aorta (Ao) and the raw pulse oximeter waveform output from the pulse oximeter (Pox). Note that there is good agreement for the arterial waveform and the pulse oximeter waveform with the left ventricular pressure.

These monitoring techniques may be used in a wide variety of settings to noninvasively measure pump function in a human or an animal, such as a race horse, a cat, a dog, an athlete, a patient at varying stage of wellness, etc.

The monitoring techniques may provide an extremely sensitive way to monitor heart function. For example, by monitoring a rate of rise or fall of an instance of a pulse-oximetry waveform, the monitoring techniques may provide a noninvasive way to monitor a rate of rise or fall of blood pressure in the left ventricle and/or blood pressure in a peripheral artery, such as: the aorta, a brachial artery, a radial artery, etc. Thus, the monitoring techniques may provide a sensitive way to measure cardiac function (e.g., the monitoring techniques may be more sensitive to a change in cardiac function than blood pressure). In some embodiments, by detecting changes when an individual exercises (such as small changes in regional function), the monitoring techniques may allow disease or changes associated with disease to be more easily detected. Notably, when an artery is occluded, a change may be detected within 5 s (as opposed to approximately 1 min. with an ECG). This may result in a significant improvement in the efficiency of an electronic device or a computer system that implements the monitoring techniques, such as: reduced processor cycles, reduced memory consumption, reduced network bandwidth to communicate data, etc.

Note that an ejection fraction may be a static value corresponding to a difference of the systolic pressure and the diastolic pressure. Moreover, the ejection fraction may be a global measurement. In contrast, the monitoring techniques may provide a more sensitive, local measurement of the total efficacy of muscle contraction of the heart. For example, the monitoring techniques may facilitate continuous, periodic or as-needed monitoring in a doctor's office, a hospital or at home relative to a baseline (which may have an unknown duration). In some embodiments, the monitoring techniques may use optical (e.g., red) light and/or infrared light, such as: light having wavelengths between 620-750 nm and/or between 700 nm-1 mm.

The noninvasive monitoring techniques may be used in: an operating room, in an intensive care unit, and/or to monitor a patient with heart disease (e.g., at home). Moreover, the monitoring techniques may be used by a medical professional (such as a physician) or by a lay-person.

While some embodiments of the monitoring techniques may use a second derivative of an instance of the pulse oximeter waveform or measurements, in other embodiments a first derivative of the instance of the pulse oximeter waveform is used. Moreover, in some embodiments, the second derivative of the instance of the pulse oximeter waveform is used when a first derivative of the instance of the pulse oximeter waveform is approximately constant (such as within 5-10% of a baseline value, e.g., a value in a preceding time interval, such as 30 s, 1 min., 5 min, 10 min. or 30 min.).

Note that the first derivative of the pressure waveform may be used because it may be most affected by the pumping action of the heart muscle and less affected by other parts of the circulatory system. It may measure the rate of pressure generation or muscle shortening from the time the muscle is stimulated to contract and the mitral valve closes, until the aortic valve opens and blood begins to escape the heart (which is sometimes referred to as ‘the period of isovolumic contraction’). In general, the things that can affect this event may be: a change in the muscle's ability to contract, an increase in blood volume returning to the heart, or a fall in the diastolic pressure. When diastolic pressure is stable and venous return to the heart is stable then changes in contractility can be very sensitively measured.

Furthermore, note that the second derivative of the pulse oximeter waveform may be very dependent on the entire circulatory system, such as: changes in volume in the circulatory system, or changes in vascular resistance or pump function. Such changes can influence the instance of the pulse oximeter waveform quite dramatically and correct analysis of the variable that is changing can be challenging. Consequently, analysis of one or more instances of the pulse oximeter waveform may involve use of a predefined analysis model (such as a neural network or a machine-learning model) that determines blood pressure from the blood-flow signal measured by the pulse oximeter.

In the present discussion, ventricular dP/dT may be a rate of pressure change with time during isovolemic contraction of the cardiac ventricles, e.g., in the period before the aortic valve and/or pulmonic valve opens, when there is no considerable change in left atrial or right atrial pressure. It may be a noninvasive technique for measuring the contractility of the left ventricle using pulse oximetry. However, in other embodiments, echocardiography may be used instead of or in addition to pulse oximetry.

In some embodiments, dP/dt may be used to predict postoperative systolic function in an individual with mitral valve regurgitation. However, the disclosed monitoring techniques may be used to monitor a variety of cardiac conditions.

For example, a continuous-wave Doppler velocity waveform of a mitral regurgitation jet may be determined from an apical four-chamber view. This may enable the measurement of instantaneous pressure gradients between the left ventricle and the left atrium. The left ventricle dP/dt may be calculated using an interval of 1-3 m/sec on the mitral regurgitation velocity spectrum.

The rate of pressure change in the right ventricle may be calculated from the tricuspid regurgitation in the same way as in the left ventricle using the mitral regurgitation jet. The exception may be that, on the right side, an interval of 1-2 m/s may be used.

Note that dP/dt may denote a rate at which left ventricular pressure rises and may indicate systolic contractile function. For example, for the left ventricle, normal may be greater than 1200 mm Hg/s, borderline may be 800-1200 mm Hg/s, reduced may be less than 800 mm Hg/s, and severely reduced may be less than 500 mm Hg/s. Alternatively, for the right ventricle, normal may be greater than 400 mm Hg/s.

We now describe embodiments of a monitoring device, an electronic device, or a computer in a computer system, which may perform at least some of the operations in the monitoring techniques. FIG. 12 presents a block diagram illustrating an example of an electronic device 1200, e.g., monitoring device 110, electronic device 112, access points 116, radio node 118, switch 128 and/or a computer or server in computer system 130, in accordance with some embodiments. For example, electronic device 1200 may include: processing subsystem 1210, memory subsystem 1212, and networking subsystem 1214. Processing subsystem 1210 includes one or more devices configured to perform computational operations. For example, processing subsystem 1210 can include one or more microprocessors, ASICs, microcontrollers, programmable-logic devices, GPUs and/or one or more DSPs. Note that a given component in processing subsystem 1210 are sometimes referred to as a ‘computation device’.

Memory subsystem 1212 includes one or more devices for storing data and/or instructions for processing subsystem 1210 and networking subsystem 1214. For example, memory subsystem 1212 can include dynamic random access memory (DRAM), static random access memory (SRAM), and/or other types of memory. In some embodiments, instructions for processing subsystem 1210 in memory subsystem 1212 include: program instructions or sets of instructions (such as program instructions 1222 or operating system 1224), which may be executed by processing subsystem 1210. Note that the one or more computer programs or program instructions may constitute a computer-program mechanism. Moreover, instructions in the various program instructions in memory subsystem 1212 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 1210.

In addition, memory subsystem 1212 can include mechanisms for controlling access to the memory. In some embodiments, memory subsystem 1212 includes a memory hierarchy that comprises one or more caches coupled to a memory in electronic device 1200. In some of these embodiments, one or more of the caches is located in processing subsystem 1210.

In some embodiments, memory subsystem 1212 is coupled to one or more high-capacity mass-storage devices (not shown). For example, memory subsystem 1212 can be coupled to a magnetic or optical drive, a solid-state drive, or another type of mass-storage device. In these embodiments, memory subsystem 1212 can be used by electronic device 1200 as fast-access storage for often-used data, while the mass-storage device is used to store less frequently used data.

Networking subsystem 1214 includes one or more devices configured to couple to and communicate on a wired and/or wireless network (i.e., to perform network operations), including: control logic 1216, an interface circuit 1218 and one or more antennas 1220 (or antenna elements). (While FIG. 12 includes one or more antennas 1220, in some embodiments electronic device 1200 includes one or more nodes, such as antenna nodes 1208, e.g., a metal pad or a connector, which can be coupled to the one or more antennas 1220, or nodes 1206, which can be coupled to a wired or optical connection or link. Thus, electronic device 1200 may or may not include the one or more antennas 1220. Note that the one or more nodes 1206 and/or antenna nodes 1208 may constitute input(s) to and/or output(s) from electronic device 1200.) For example, networking subsystem 1214 can include a Bluetooth™ networking system, a cellular networking system (e.g., a 3G/4G/5G network such as UMTS, LTE, etc.), a USB networking system, a networking system based on the standards described in IEEE 802.11 (e.g., a Wi-Fi® networking system), an Ethernet networking system, and/or another networking system.

Networking subsystem 1214 includes processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for the network system. Moreover, in some embodiments a ‘network’ or a ‘connection’ between electronic devices does not yet exist. Therefore, electronic device 1200 may use the mechanisms in networking subsystem 1214 for performing simple wireless communication between electronic devices, e.g., transmitting advertising or beacon frames and/or scanning for advertising frames transmitted by other electronic devices.

Within electronic device 1200, processing subsystem 1210, memory subsystem 1212, and networking subsystem 1214 are coupled together using bus 1228. Bus 1228 may include an electrical, optical, and/or electro-optical connection that the subsystems can use to communicate commands and data among one another. Although only one bus 1228 is shown for clarity, different embodiments can include a different number or configuration of electrical, optical, and/or electro-optical connections among the subsystems.

In some embodiments, electronic device 1200 includes a display subsystem 1226 for displaying information on a display, which may include a display driver and the display, such as a liquid-crystal display, a multi-touch touchscreen, etc. Moreover, electronic device 1200 may include a user-interface subsystem 1230, such as: a mouse, a keyboard, a trackpad, a stylus, a voice-recognition interface, and/or another human-machine interface.

Electronic device 1200 can be (or can be included in) any electronic device with at least one network interface. For example, electronic device 1200 can be (or can be included in): a desktop computer, a laptop computer, a subnotebook/netbook, a server, a supercomputer, a tablet computer, a smartphone, a smartwatch, a pulse oximeter, a cellular telephone, a consumer-electronic device, a portable computing device, communication equipment, a monitoring device and/or another electronic device.

Although specific components are used to describe electronic device 1200, in alternative embodiments, different components and/or subsystems may be present in electronic device 1200. For example, electronic device 1200 may include one or more additional processing subsystems, memory subsystems, networking subsystems, and/or display subsystems. Additionally, one or more of the subsystems may not be present in electronic device 1200. Moreover, in some embodiments, electronic device 1200 may include one or more additional subsystems that are not shown in FIG. 12. Also, although separate subsystems are shown in FIG. 12, in some embodiments some or all of a given subsystem or component can be integrated into one or more of the other subsystems or component(s) in electronic device 1200. For example, in some embodiments program instructions 1222 are included in operating system 1224 and/or control logic 1216 is included in interface circuit 1218.

Moreover, the circuits and components in electronic device 1200 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit may implement some or all of the functionality of networking subsystem 1214 and/or electronic device 1200. The integrated circuit may include hardware and/or software mechanisms that are used for transmitting signals from electronic device 1200 and receiving signals at electronic device 1200 from other electronic devices. Aside from the mechanisms herein described, radios are generally known in the art and hence are not described in detail. In general, networking subsystem 1214 and/or the integrated circuit may include one or more radios.

In some embodiments, an output of a process for designing the integrated circuit, or a portion of the integrated circuit, which includes one or more of the circuits described herein may be a computer-readable medium such as, for example, a magnetic tape or an optical or magnetic disk or solid state disk. The computer-readable medium may be encoded with data structures or other information describing circuitry that may be physically instantiated as the integrated circuit or the portion of the integrated circuit. Although various formats may be used for such encoding, these data structures are commonly written in: Caltech Intermediate Format (CIF), Calma GDS II Stream Format (GDSII), Electronic Design Interchange Format (EDIF), OpenAccess (OA), or Open Artwork System Interchange Standard (OASIS). Those of skill in the art of integrated circuit design can develop such data structures from schematics of the type detailed previously and the corresponding descriptions and encode the data structures on the computer-readable medium. Those of skill in the art of integrated circuit fabrication can use such encoded data to fabricate integrated circuits that include one or more of the circuits described herein.

While some of the operations in the preceding embodiments were implemented in hardware or software, in general the operations in the preceding embodiments can be implemented in a wide variety of configurations and architectures. Therefore, some or all of the operations in the preceding embodiments may be performed in hardware, in software or both. For example, at least some of the operations in the monitoring techniques may be implemented using program instructions 1222, operating system 1224 (such as a driver for interface circuit 1218) or in firmware in interface circuit 1218. Thus, the monitoring techniques may be implemented at runtime of program instructions 1222. Alternatively or additionally, at least some of the operations in the monitoring techniques may be implemented in a physical layer, such as hardware in interface circuit 1218.

In the preceding description, we refer to ‘some embodiments.’ Note that ‘some embodiments’ describes a subset of all of the possible embodiments, but does not always specify the same subset of embodiments. Moreover, note that the numerical values provided are intended as illustrations of the monitoring techniques. In other embodiments, the numerical values can be modified or changed.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. An integrated circuit, wherein the integrated circuit that monitors blood pressure in vasculature of an individual, wherein the integrated circuit is configured to:
monitor blood flow of the individual;
calibrate the blood flow based at least in part on a predefined or predetermined comparison of the blood flow and blood pressure in the individual's vasculature; and
provide the calibrated blood flow of the individual as a function of time, wherein the calibrated blood flow indicates at least the blood pressure or a blood-pressure reading for the individual.

2. The integrated circuit of claim 1, wherein, during the monitoring, the electronic device is coupled or attached to the individual.

3. The integrated circuit of claim 1, wherein the monitoring of the blood flow is performed continuously, periodically or as needed.

4. The integrated circuit of claim 1, wherein the monitoring of the blood flow is performed using a pulse oximeter.

5. The integrated circuit of claim 4, wherein, during the monitoring, the electronic device is configured to acquire an instance of a pulse oximeter waveform of the individual.

6. The integrated circuit of claim 1, wherein the providing comprises displaying the calibrated blood flow of the individual.

7. The integrated circuit of claim 1, wherein the calibrated blood flow is sinusoidal, with each pulse in the calibrated blood flow having a peak amplitude A, and a time interval Δt between the peak amplitude of a given pulse and the peak amplitude of an immediately preceding pulse; and
wherein the electronic device is configured to:
compute a heart rate corresponding to Δt, a blood-flow volume corresponding to Δt and A, or both; and
provide the computed heart rate and/or blood-flow volume.

8. The integrated circuit of claim 1, wherein the providing of the calibrated blood flow comprises at least selectively presenting changes in the blood pressure in the context of either a first operational state in which Δt is constant or a second operational state in which Δt is variable.

9. The integrated circuit of claim 1, wherein the electronic device is configured to perform a calibration comprising measuring an instance of the comparison of the blood flow and the blood pressure using, at least in part, a sphygmomanometer.

10. The integrated circuit of claim 9, wherein the instance of the comparison is measured at a steady-state condition for the relationship between a measurement of the blood flow and a measurement of the blood pressure.

11. The integrated circuit of claim 10, wherein the measurement of the blood pressure comprises a difference between a systolic pressure and a diastolic pressure.

12. The integrated circuit of claim 10, wherein the measurement of the blood flow and the measurement of the blood pressure are obtained concurrently.

13. The integrated circuit of claim 10, wherein the predefined or determined comparison of the blood flow and the blood pressure correlates or includes changes in the blood flow with changes in the blood pressure.

14. An electronic device, comprising:
a light source; and
at least an integrated circuit, coupled to the light source, configured to:
monitor blood flow of an individual;
calibrate the blood flow based at least in part on a predefined or predetermined comparison of the blood flow and blood pressure in the individual's vasculature; and
provide the calibrated blood flow of the individual as a function of time, wherein the calibrated blood flow indicates at least the blood pressure or a blood-pressure reading for the individual.

15. The electronic device of claim 14, wherein the electronic device comprises a pulse oximeter.

16. The electronic device of claim 14, wherein the calibrated blood flow is sinusoidal, with each pulse in the calibrated blood flow having a peak amplitude A, and a time interval Δt between the peak amplitude of a given pulse and the peak amplitude of an immediately preceding pulse; and
wherein the method comprises:
computing a heart rate corresponding to Δt, a blood-flow volume corresponding to Δt and A, or both; and
providing the computed heart rate and/or blood-flow volume.

17. The electronic device of claim 14, wherein the method comprises performing a calibration comprising measuring an instance of the comparison of the blood flow and the blood pressure using, at least in part, a sphygmomanometer.

18. The electronic device of claim 17, wherein the instance of the comparison is measured at a steady-state condition for the relationship between a measurement of the blood flow and a measurement of the blood pressure.

19. A method for providing calibrated blood flow of an individual, comprising:
by an electronic device:
monitoring blood flow of the individual;
calibrating the blood flow based at least in part on a predefined or predetermined comparison of the blood flow and blood pressure in the individual's vasculature; and
providing the calibrated blood flow of the individual as a function of time, wherein the calibrated blood flow indicates at least the blood pressure or a blood-pressure reading for the individual.

20. The method of claim 19, wherein the electronic device comprises a pulse oximeter.
